(19) United States
(12) Patent Application Publication
(10) Pub. No.: US 2008/0267346 A1
(43) Pub. Date: Oct. 30, 2008

(54) X-Ray Imaging Apparatus and Method

(75) Inventor: Tiemen Poorter, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/913,326

(22) PCT Filed: Apr. 28, 2006
(86) PCT No.: PCT/IB2006/051335

§ 371 Date: Nov. 1, 2007

(30) Foreign Application Priority Data
May 4, 2005 (EP) 05103756.2

(51) Int. Cl. A61B 6/00; G01N 23/087

(52) U.S. Cl. 378/16

(57) ABSTRACT

The present invention relates to an X-ray imaging apparatus and a corresponding X-ray imaging method, in particular for X-ray fluoroscopy and for an application where a characteristic feature shall be extracted from acquired X-ray images. In order to reduce the total X-ray dose to which a patient is exposed for acquiring a series of X-ray images and, nevertheless, to allow the extraction of small characteristic features with high certainty, an X-ray imaging apparatus is proposed comprising: an imaging unit comprising an X-ray source (2) and an X-ray detector (4) for forming a series of X-ray images of an object (P) including a characteristic feature, a feature extraction unit (6) for extracting said characteristic feature from said series of X-ray images, and a control unit (7) for controlling the X-ray dose to which the object (P) is exposed for forming said series of X-ray images by controlling said X-ray source (2) such that the X-ray dose per X-ray image is larger for a first number of images than for the remaining number of images of said series.

The present invention relates to an X-ray imaging apparatus and a corresponding X-ray imaging method, in particular for—but not limited to—X-ray fluoroscopy. Further, the present invention relates to a computer program for controlling said X-ray imaging apparatus. The invention can, preferably, be applied in the medical field of cardiology, in particular for extracting, registering and enhancing thin objects of interest, hereinafter generally called characteristic features, such as stents and vessel walls in arteriograms.

A medical viewing system and a method for detecting and enhancing structures in noisy images are disclosed in WO 03/043516 A2 and WO 03/04263 A2. In particular, a medical viewing system is disclosed for displaying a sequence of images of a medical intervention that comprises moving and/or positioning a tool in a body organ, which tool is carried by a support to which at least one marker is attached at a predetermined location with respect to the tool. Enabling the processing of medical images in order to be used during the intervention phase, the medical viewing system disclosed in WO 03/043516 A2 comprises means for acquiring the sequence of images and for processing said images during the medical intervention, extracting means for automatically extracting at least one marker that is attached to the tool support and that neither belongs to the tool nor to the body organ, yielding the marker location information, computing means for automatically deriving the tool location information from the marker location information, and enhancing means for improving the visibility of the tool and/or the body organ in order to check whether the medical intervention stages are successfully carried out. Furthermore, details of particular applications, such as a stent implantation, and details of the extraction means and the method carried out by such means can be found in these documents, to which herewith reference is made and which are herein incorporated by reference.

When specific small features, such as medical tools or small image objects, need to be extracted from X-ray images, the signal to noise ratio in these X-ray images needs to be rather good. Improving the signal to noise ratio in medical X-ray images generally requires a relatively high exposure dose (X-ray dose) to generate these images. However, in X-ray fluoroscopy, which is often applied in cardiovascular imaging, a series of X-ray images needs to be acquired subsequently, which series may consist of 10 to several hundreds of X-ray images. The patient would thus be exposed to a large total X-ray dose.

It is an object of the present invention to provide an X-ray imaging apparatus and a corresponding X-ray imaging method by which the total X-ray dose to which a patient is exposed in case a series of X-ray images shall be acquired can be reduced, but which nevertheless allow the extraction of small features with certainty.

The object is achieved according to the present invention by an X-ray imaging apparatus as claimed in claim 1 comprising:

an imaging unit comprising an X-ray source and an X-ray detector for forming a series of X-ray images of an object including a characteristic feature,

a feature extraction unit for extracting said characteristic feature from said series of X-ray images, and

a control unit for controlling the X-ray dose to which the object is exposed for forming said series of X-ray images by controlling said X-ray source such that the X-ray dose per X-ray image is larger for a first number of images than for the remaining number of images of said series.

A corresponding X-ray imaging method is defined in claim 11. A computer program comprising program code means for controlling the X-ray imaging apparatus according to said method is defined in claim 12. Preferred embodiments of the invention are defined in the dependent claims.

The invention is based on the idea that the signal to noise ratio that is required to find certain characteristic features in X-ray images is lower when the same characteristic features have already been detected in previous X-ray images of the same acquisition run. Thus, it has been found that only for the first images of an image run for acquiring a series of X-ray images a high signal to noise ratio is required allowing the extraction of the desired characteristic feature(s) with high certainty, but that for the subsequent images the signal to noise ratio can be significantly reduced. Thus, when using a feature extraction method as, for instance, described in the above mentioned international patent applications, in a medical imaging run, the X-ray dose per frame is set at a higher level for a few images to properly find the relevant characteristic feature(s) in the image. When the feature(s) have been detected, all other images in the same run will be made with a strongly reduced X-ray dose level. This reduces the total X-ray dose to which the patient is exposed, while at the same time the quality of the feature extraction operation is significantly improved due to the higher initial X-ray dose.

According to a preferred embodiment the first number for which the X-ray dose is set at a high level is in the range from 1 to 20, in particular from 2 to 5, or in the range from 1 to 10%, in particular from 1 to 2%, of the total number of images of said series. Thus, in view of the possibly large number of images acquired in one series, this is only a very small number but is sufficient to extract the desired characteristic feature with certainty.

According to a further preferred embodiment the characteristic feature is a medical tool positioned inside the object, in particular a stent, catheter or guide wire. Preferably, said medical tool is provided with at least one detectable marker. These markers, which neither belong to the organ nor to the medical tool, preferably have a specific easily recognizable shape and/or are made of a material highly contrasted in the images, so that they are easy to extract. The application and extraction of such medical tools provided with a marker is described in detail in the above mentioned two international patent applications to which reference is made herewith.

It should be noted that the extraction of position information is not limited to the use of markers attached to specific medical tools. It is possible to track parts of a specific tool and possibly parts of anatomy with sufficient contrast, as well. For instance, according to one embodiment features of objects, that show sufficient contrast to their surrounding, can be tracked and extracted by the feature extraction unit.

In an advantageous embodiment it is proposed that the X-ray dose is controlled such that the X-ray dose is kept at a high level until the characteristic feature has been extracted by said feature extraction unit. Thus, immediately after a successful extraction, the X-ray dose will be decreased in order to avoid, in view of feature extraction, an unnecessary high X-ray dose.

Preferably, the control unit is adapted for controlling the X-ray dose such that the X-ray dose per image is set to a high level in the range from 20 to 200%, in particular from 50 to 100%, above the normal level for said first number of images and, further preferably, for controlling the X-ray dose such that the X-ray dose per image is set to a low level in the range from 10 to 90%, in particular from 20 to 50%, of the normal level for said remaining number of images. Thus, in view of the low number of images for which the X-ray dose is set to a high level, a strong reduction of the total X-ray dose can be achieved.

The “normal” dose level can be regarded as the dose which provides an acceptable chance of finding the desired feature within the capabilities of the image processing capabilities at that moment in time. Of course, this dose level is dependent on the available imaging system and it is expected that this dose level will be lower in the future. If e.g. the chance of finding the desired feature is 90% at a certain dose level per image this chance will increase with increasing dose per image. Increasing the dose per image to a level such that the chance of finding the feature is e.g. >99% would require a considerably higher dose which would be unacceptable for longer periods.

Thus, the “normal” level may be considered as the level at which a certain image processing technology for feature extraction may detect a certain feature with a certain contrast and geometrical structure with a certain chance of e.g. >50%-95% and in particular with >70%-90%.

As mentioned, the invention is preferably applied for forming a series of fluoroscopy X-ray images from a fixed projection direction, called angiograms. The objects of interests are preferably organs such as arteries and tools such as balloons or stents which are observed during a medical intervention called angioplasty. However, of course, the invention may be applied in other fields as well for other applications and for other objects of interest where other characteristic features need to be extracted.

In a particular embodiment the feature extraction unit comprises a detection unit for automatically detecting said characteristic feature, in particular at least one marker that is attached to said characteristic feature, and a computing unit for automatically deriving location information of said characteristic feature from said detection.

The invention will now be explained in more detail with reference to the drawings in which

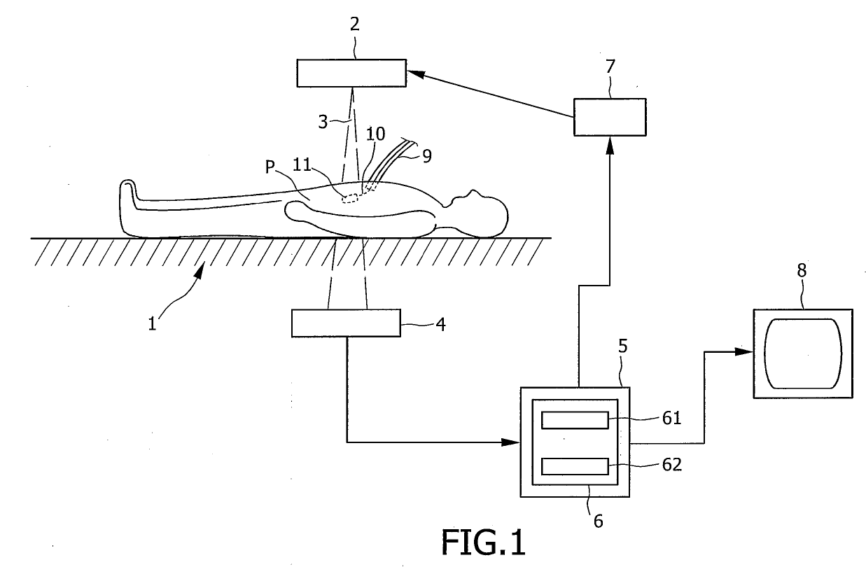

FIG. 1 schematically shows an X-ray imaging apparatus according to the present invention and

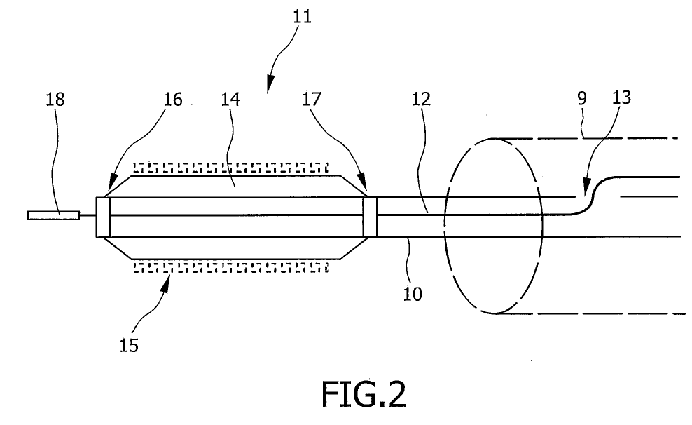

FIG. 2 shows a medical tool to be extracted from X-ray images.

In the schematic diagram of FIG. 1 an X-ray imaging apparatus according to the present invention is shown. A patient P lies on a patient table 1 so that a series of X-ray images of the patient P can be formed, for instance a series of fluoroscopy images of the coronary arteries of the patient during a medical intervention. For forming an X-ray image an X-ray generator 2 emits X-rays, for instance at regular intervals or upon user interaction, so that an X-ray beam 3 passes through the patient and is received by an X-ray detector 4. Said detector 4 is connected to processing means 5 for forming the X-ray images which may be displayed on a display screen 8. For controlling the emission of the X-rays by the X-ray generator 2 a control unit 7 is provided.

As an example of an application, in which the present invention can preferably be applied, a stent implantation shall be considered more in detail in the following. A stent implantation is a medical intervention that usually comprises several stages for enlarging an artery at the location of a lesion called stenosis.

A medical tool used in such a stent implantation is shown in more detail in FIG. 2. It comprises a catheter 9 in which a guide wire 12 is guided which itself is used for guiding a monorail 10 and which passes through the opening 13 of the monorail 10. At the extremity of the monorail 10 a balloon 14 is wrapped around the monorail 10 around which a stent 15 is wrapped. Further, markers 16, 17 and 18 can be provided, for instance at a given position with respect to the balloon 14 on the monorail 10 or the extremity of the guide wire 12, which markers are easily detectable and extractable from X-ray images.

In a preliminary stage of the stent implantation, the practitioner localizes a stenosis in a patient's artery as best as possible in medical images. Thereafter a number of further stages follow, comprising the steps of:

introducing the thin guide wire 10 using a catheter 9 and introducing the monorail 10 which is guided by said guide wire 10 and having a first balloon wrapped around its extremity;

positioning said balloon in the artery portion at the stenosis location;

inflating the first balloon;

removing the first balloon;

introducing a second monorail with a second balloon wrapped around its extremity and with a stent around said second balloon;

positioning said second balloon with the stent at the location of the stenosis in the previously expanded lumen of the artery;

inflating the second balloon to expand the coil forming the stent; and

removing the second balloon, the second monorail, the guide wire and the catheter leaving the expanded stent as a permanent implant.

Such a medical intervention (called angioplasty) is difficult to carry out due to badly contrasted medical images where the medical tools are hardly distinguishable on a noisy background and are, moreover, subjected to motions. The processing means 5 thus include a feature extraction unit 6 which is adapted for automatically and accurately localizing specific features in the X-ray images. Such a feature extraction unit and the use thereof for stent implantation is described in more detail in WO 03/043516 A2 and WO 03/045263 A2 to which reference is herewith made. Said feature extraction unit 6 mainly comprises a detection unit 61 for automatically detecting a characteristic feature in said X-ray images and a computing unit 62 for automatically deriving a location information of said characteristic feature from said detection.

As noted above the feature extracted by said feature extraction unit 6 can not only be one or more markers attached to a medical tool, but also a characteristic part of the medical tool itself or a characteristic feature of the object, if said characteristic part or said characteristic feature show sufficient contrast in the X-ray images allowing for an automatic feature extraction.

It has been recognized according to the invention that it is sufficient to have a high signal to noise ratio in a first view X-ray images in order to extract from said images the desired characteristic feature, for instance the at least one marker which allows the determination of the medical tool's position. Thereafter, such a high signal to noise ratio is no longer required, but a lower signal to noise ratio is sufficient to find the same features in subsequent images. Thus, the control unit 7 provided according to the present invention is adapted for controlling the X-ray dose to which the patient P is subjected when the X-ray images are acquired such that the X-ray dose is larger during the acquisition of the first view images leading to a high signal to noise ratio while the X-ray dose for acquiring the remaining number of X-ray images is strongly reduced. For instance, in an imaging run which may comprise several tens to several hundreds of images in a fluoroscopy application, the first 2 to 5 images may be acquired with a high X-ray dose, for instance in a range from 50 to 100% above the normal level, and thereafter the X-ray dose is set to a level in the range from 20 to 50% of the normal level for acquiring all remaining images of said imaging run.

There may also be provided a coupling between the control unit 7 and the feature extraction unit 6 by which the feature extraction unit can transmit an indication to the control unit that the desired characteristic feature has been extracted so that immediately thereafter the control unit can reduce the X-ray dose from a high to a low level.

According to the present invention the feature extraction unit uses the information about the location of the characteristic feature obtained in the first view images for finding the same characteristic feature in the remaining number of images which have been acquired with lower X-ray dose. For this purpose a marker tracking method can be used: When a characteristic feature (or marker) has been detected in one image it is possible to predict its position in subsequent images from motion estimation from previous images. The predicted position of the characteristic feature (or marker) in the subsequent images is then used as a method to strongly reduce the number of possible locations of the position of the characteristic feature (or marker) in these next images. In this way the chance of finding the marker or feature is strongly increased. This leads to a method that delivers good tracking quality even at the reduced S/N ratio in the images from the reduced X-ray dose per image.

By use of the invention the total X-ray dose to which the patient is subjected during the medical imaging run can be strongly reduced while at the same time the quality of the feature extraction operation can be improved since the desired characteristic feature can be extracted with high certainty from the first view images.

One could also say that the essence of the invention is that once the feature has been recognized it is much easier to find it in subsequent images so that the dose can be reduced to a dose considerably lower than the level for a chance of e.g. 90% of finding the feature for the first time. So according to the invention a small number of images could be made with a dose per frame such that the chance of finding it in any image is >99% while the dose can be reduced to a much lower level (at which the chance of finding the feature in any image would, for instance, be <20%) for the subsequent images because the feature is known in the previous image and can be detected at this lower dose level with the knowledge from the prior image(s) reliably.

It is also clear that the dose level at which the technology of a certain company is able to find certain features will be different from that of other companies and this will also vary over time. It will also depend on the type of feature (e.g. size of the stent) for which the operation will be performed.

1. X-ray imaging apparatus comprising:
an imaging unit comprising an X-ray source (2) and an X-ray detector (4) for forming a series of X-ray images of an object (P) including a characteristic feature,
a feature extraction unit (6) for extracting said characteristic feature from said series of X-ray images, and
a control unit (7) for controlling the X-ray dose to which the object (P) is exposed for forming said series of X-ray images by controlling said X-ray source (2) such that the X-ray dose per X-ray image is larger for a first number of images than for the remaining number of images of said series.

2. X-ray imaging apparatus as claimed in claim 1, wherein said first number is in the range from 1 to 20, in particular from 2 to 5.

3. X-ray imaging apparatus as claimed in claim 1, wherein said first number is in the range from 1 to 10%, in particular from 1 to 2%, of the total number of images of said series.

4. X-ray imaging apparatus as claimed in claim 1, wherein said characteristic feature is a medical tool (11) positioned inside the object (P), in particular a stent (15), catheter (9) or guide wire (12).

5. X-ray imaging apparatus as claimed in claim 4, wherein said medical tool (11) is provided with a detectable marker (16, 17, 18).

6. X-ray imaging apparatus as claimed in claim 1, wherein said control unit (7) is adapted for controlling the X-ray dose such that the X-ray dose is kept at a high level until the characteristic feature has been extracted by said feature extraction unit (6).

7. X-ray imaging apparatus as claimed in claim 1, wherein said control unit (7) is adapted for controlling the X-ray dose such that the X-ray dose per image is set to a high level in the range from 20 to 200%, in particular from 50 to 100%, above the normal level for said first number of images.

8. X-ray imaging apparatus as claimed in claim 1, wherein said control unit (7) is adapted for controlling the X-ray dose such that the X-ray dose per image is set to a low level in the range from 10 to 90%, in particular from 20 to 50%, of the normal level for said remaining number of images.

9. X-ray imaging apparatus as claimed in claim 1, wherein said imaging unit (2, 4) is adapted for forming a series of fluoroscopy X-ray images from a fixed projection direction.

10. X-ray imaging apparatus as claimed in claim 1, wherein said feature extraction unit (6) comprises a detection unit (61) for automatically detecting said characteristic feature, in particular at least one marker that is attached to said characteristic feature, and a computing unit (62) for automatically deriving location information of said characteristic feature from said detection.

11. X-ray imaging method comprising the steps of:
forming a series of X-ray images of an object including a characteristic feature by an imaging unit comprising an X-ray source and an X-ray detector,
extracting said characteristic feature from said series of X-ray images, and
controlling the X-ray dose to which the object is exposed for forming said series of X-ray images by controlling said X-ray source such that the X-ray dose per X-ray image is larger for a first number of images than for the remaining number of images of said series.

12. Computer program comprising program code means for controlling the X-ray imaging apparatus as claimed in claim 1 according to the method comprising the steps of:
forming a series of X-ray images of an object including a characteristic feature by an imaging unit comprising an X-ray source and an X-ray detector,
extracting said characteristic feature from said series of X-ray images, and
controlling the X-ray dose to which the object is exposed for forming said series of X-ray images by controlling said X-ray source such that the X-ray dose per X-ray image is larger for a first number of images than for the remaining number of images of said series when said computer program is run on a computer.
